(19) United States
(12) Patent Application Publication
(10) Pub. No.: US 2023/0218014 A1
(43) Pub. Date: Jul. 13, 2023

(54) Positional Therapy Maternity Belt

(71) Applicant: Shiphrah Biomedical Inc., Toronto (CA)

(72) Inventors: Allan Kember, Toronto (CA); Niema Mohammad, Aurora (CA); Payal Mohini, Mississauga (CA); Jane Warland, Yokine (AU); Ningshen Feng, Burnaby (CA)

(73) Assignee: Shiphrah Biomedical Inc., Toronto, ON (CA)

(21) Appl. No.: 18/000,413

(22) PCT Filed: Jun. 9, 2021
(86) PCT No.: PCT/IB2021/000390

§ 371 Date: Dec. 1, 2022

Related U.S. Application Data
(60) Provisional application No. 63/038,209, filed on Jun. 12, 2020.

(51) Int. Cl. A41C 1/10; A47C 20/02

(52) U.S. Cl. CPC A41C 1/10; A47C 20/025; A61F 5/56

(57) ABSTRACT

Implemented is a positional therapy maternity belt that interoperates with and leverages multiple removably attachable pillows and other components to facilitate enhanced and effortless positional therapy for a user during pregnancy. The maternity belt comes equipped with a right and left band that wraps around the user's waist. In between the bands and at a central region of the belt is overlaid a loop fastener to enable additional cushion and component attachment thereto. The left and right bands are comprised elastic strapping faced with 85% nylon and 15% polyester knit on the side that faces against the user's body (inside/interior side) and a 100% nylon loop face on the side that faces away from the user's body (outside/exterior side).

### CROSS-REFERENCE TO RELATED APPLICATIONS

This Non-Provisional application claims the benefit of and priority to PCT Application Serial No. PCT/IB2021/000390, filed Jun. 9, 2021, entitled “Positional Therapy Maternity Belt,” which claims the benefit of and priority to U.S. Provisional Patent Application Ser. No. 63/038,209, entitled “Wearable Girdle Used for Musculoskeletal Support and Positional Therapy in Pregnant Women,” filed Jun. 12, 2020, the entire contents of both applications of which are hereby incorporated herein by reference.

### BACKGROUND

In obstetrics, it is well-known that the supine position in pregnant women alters maternal cardiovascular parameters, particularly during labor. Recent research has identified the maternal supine sleeping position as a possible novel modifiable risk factor for stillbirth and low birth weight. It has also been demonstrated that low birthweight mediates the effects of supine sleep on stillbirth, it is estimated that 6% of stillbirths and up to 17% of low birth weights may be averted through modification of this novel risk factor.

Furthermore, the physiological and hormonal changes associated with pregnancy result in changes in sleep architecture, which place pregnant women at higher risk for the development of sleep-disordered breathing, which refers to a wide spectrum of sleep-related breathing abnormalities including increased upper airway resistance (snoring) and obstructive sleep apnea-hypopnea syndrome. The incidence of both snoring and obstructive sleep apnea has been shown to be increased in pregnant women, which may further put the fetus at risk. Indeed, the prevalence of obstructive sleep apnea in the non-pregnant population of reproductive age women is estimated to be 0.7-6.5%. However, in the 1^{st} and 3^{rd} trimesters of pregnancy, the prevalence is 8.4 and 19.7%, respectively. Positional therapy is a simple, non-invasive, inexpensive, long-established, safe, and effective intervention for preventing people with positional-dependent snoring or mild to moderate obstructive sleep apnea from sleeping supine—a position that exacerbates their condition.

There are several techniques used for positional therapy such as positional alarms, verbal instructions, special pillows, tennis balls, vests, and “shark fins”. The latter four techniques prevent the wearer from lying supine in a passive manner by physically preventing the wearer from turning into a supine position.

Some pregnant women sleep with many pillows supporting their body, including a pillow behind their back to avoid the supine position. Maternal body pillows, regular pillows, and pelvic belts (lumbar support) have been used by pregnant women during sleep without reports of serious adverse effects for the mother or neonate. Asking women to sleep on their left increases the percentage of left sided sleep to approximately 60% of the night; however, this may come at a cost of reduced sleep duration (which is known to be detrimental), perhaps due to the feeling they need to make a conscious effort to maintain sleep position.

### SUMMARY

Implemented is a positional therapy maternity belt that interoperates with and leverages multiple removably attachable pillows and other components to facilitate enhanced and effortless positional therapy for a user during pregnancy. The maternity belt comes equipped with a right and left band that wraps around the waist of the user. The left and right bands are comprised of elastic strapping faced with 85% nylon and 15% polyester knit on the inside/interior side and a 100% nylon loop face on the outside/exterior side. In between the bands and at a central region of the belt is a rectangular central section overlaid with a non-stretch loop fastener in a configuration that allows for breathability.

The central section of the maternity belt utilizes a rear hook-and-loop fastener. Multiple and various types of pillows and other components can removably attach to the central section to provide additional and differing comfort and support to the user. Exemplary pillows and components include a right-angle triangle wedge-shaped pillow, an isosceles triangle wedge-shaped pillow, a bean bag pillow, an air chamber attachment, a rectangular memory foam pillow with embedded firm shapes, a trapezoidal-shaped pillow, and a hot or cold thermal pack, among other components. All of the various pillows can be switched on and off by the user to enable the user to select the pillow or component that is ideal for their sleep position, and namely to avoid the supine sleep position.

The maternity belt is designed for adjustability and comfort. The belt permits maximum adjustment for a comfortable and secure fit and enabling easy removal with a stretch looped belt surface and hook-and-loop attachment.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure. These and various other features will be apparent from a reading of the following Detailed Description and a review of the associated drawings.

### DESCRIPTION OF THE DRAWINGS

FIGS. 1 and 2 show illustrative representations of the maternity belt from an exterior and interior view, respectively;

FIG. 3 shows an illustrative series of representations of a user putting on the maternity belt;

FIG. 4 shows an illustrative representation of an air chamber and its cover;

FIG. 5 shows an illustrative representation in which the air chamber is secured and in use by a user;

FIGS. 6A and 6B show illustrative representations of an isosceles wedge attachment;

FIG. 7 shows an illustrative representation in which the isosceles wedge is secured and in use by the user;

FIG. 8 shows an illustrative representation of a bean bag pillow;

FIG. 9 shows an illustrative representation in which the bean bag pillow is attached to the maternity belt;

FIGS. 10A and 10B show illustrative representations of a wedge-shaped pillow;

FIG. 11 shows illustrative representations in which the wedge-shaped pillow is secured and in use by the user;

FIG. 12 shows an illustrative representation of a temperature (e.g., cooling or heating) pack

FIG. 13 shows illustrative representations in which the temperature pack is secured and in use.

FIG. 14 shows an illustrative representation of a trapezoidal side pillow;

FIG. 15 shows an illustrative representation in which the trapezoidal side pillow is in use;

FIG. 16 shows an illustrative representation of a rectangular memory foam pillow with embedded firm shapes; and

FIG. 17 shows an illustrative representation in which a rectangular memory foam pillow with embedded firm shapes is in use.

Like reference numerals indicate like elements in the drawings. Elements are not drawn to scale unless otherwise indicated.

### DETAILED DESCRIPTION

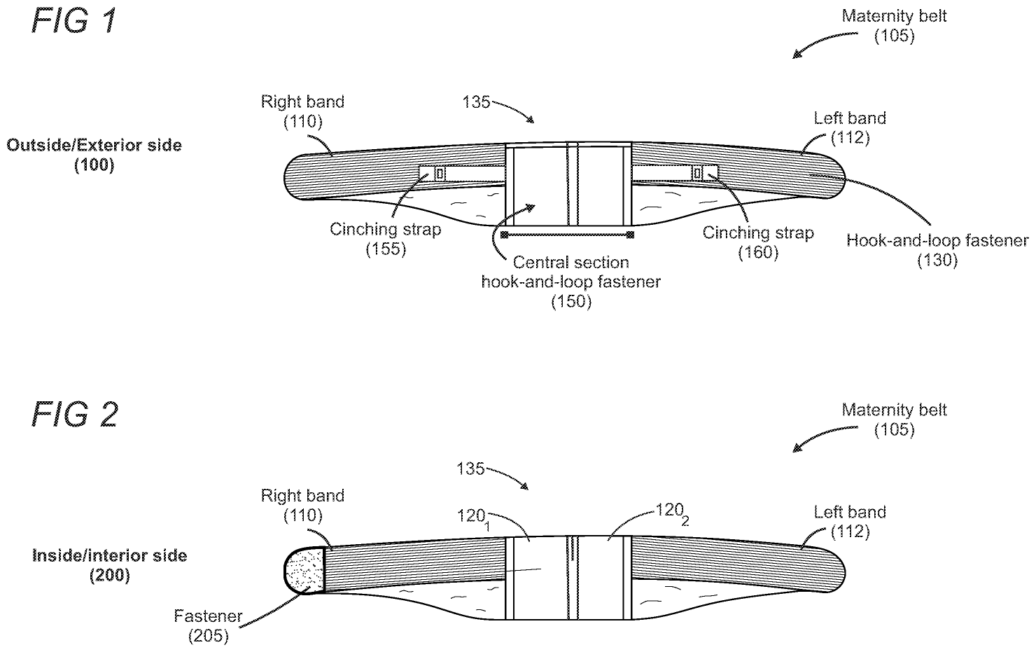

FIGS. 1 and 2 show illustrative representations in which a maternity belt 105 includes various components to aid in positional therapy for a user. The maternity belt includes a right band 110 and a left band 112 positioned on opposing sides of the maternity belt. The drawings and description herein characterize the right and left bands from the perspective of a wearer of the maternity belt. The left and right bands are comprised of elastic strapping faced with 85% nylon and 15% polyester knit on the side that faces against the user's body (inside/interior side) and a 100% nylon loop face on the side that faces away from the user's body (outside/exterior side). The bands are approximately twenty inches in length and four inches wide. However, the specific sizes and shapes may vary depending on a given implementation and may be longer, shorter, or thicker than the example provided. Furthermore, the fabric may be configured as breathable, antimicrobial, and anti-odor.

The cinching straps 155, 160 overlap with respective left and right bands 110, 112 and can be utilized to adjust the tightness of the maternity belt 105. Each cinching strap is attached, such as stitching, to the edges of the central section 135. The cinching straps are at least partially comprised of an elastic material (e.g., elastic, elastane, stretch yarn, stretch polyester, stretch nylon, etc.) to enable a user to pull and tighten or release and loosen depending on the scenario. For example, the cinching straps may be beneficial to apply the correct level of radially-directed compression without adjusting the left and right bands. Furthermore, when the maternity belt is in use when the user is laying down, the user's abdomen and overall body shift, which can cause loosening of the belt. The cinching straps can then be used to tighten and adjust the belt to have a snug fit.

An exterior side 100 of the maternity belt 105 includes a central section 135 positioned between the right and left bands 110, 112. The exterior side of the left band includes a fastener 130, and an interior side 200 of the right band includes a corresponding fastener 205. Specifically, the left band's entire exterior surface area is comprised of a fastening mechanism, such as a hook-and-loop fastener 130, and the right band includes a corresponding fastener 205 near its distal end. This arrangement enables the user to easily adjust the right band's fastener to any location on the left band. The right band's fastener overlaps and engages with the exterior side of the left band when the user wears the belt by first wrapping the left band toward their abdomen and then pulling over the right band. The fasteners in this implementation may be hook-and-loop fasteners (e.g., Velcro®), but other types of fasteners are also possible, such as snaps or friction fits, a buckle, buttons, etc.

The exterior side 100 of the central section is overlaid with a non-stretch loop fastener 150 to enable additional cushion and component attachment thereto, as discussed in greater detail below. As shown in FIG. 1, the loop fastener may extend entirely across the central section. The fastener may likewise extend from the top of the central section to the bottom (not shown).

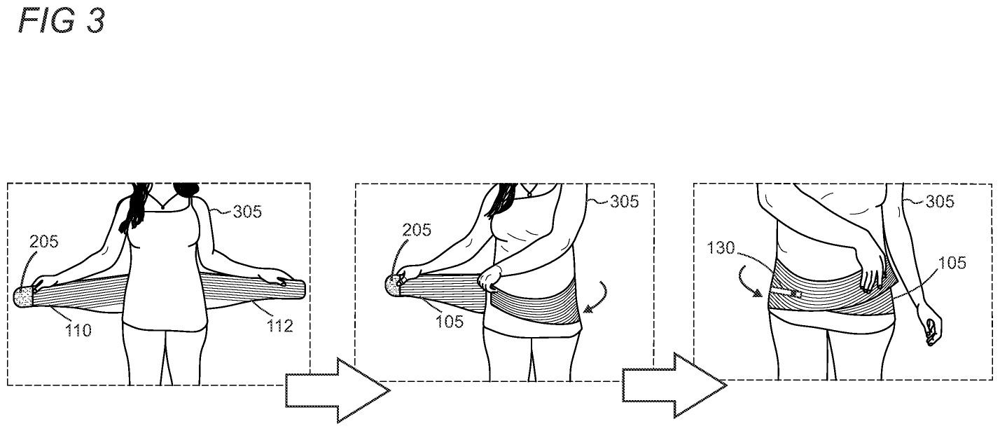

FIG. 3 shows an illustrative representation in which a user 305 puts on the maternity belt 105. First, the left band wraps around the left side of the user's waist from approximately the area of the left posterolateral iliac crest around the front of the user's waist to approximately the area of the right anterior superior iliac spine. Next, the right band wraps around the right side of the user's waist from approximately the area of the right posterolateral iliac crest around the front of the user's waist and overlying the left band to approximately the area of the left anterior superior iliac spine where the hook fastener 205 engages the exterior side of the left band. Together with the central section dimensions, the left and right bands accommodate a user's waist size of approximately 28 inches up to 60 inches in circumference.

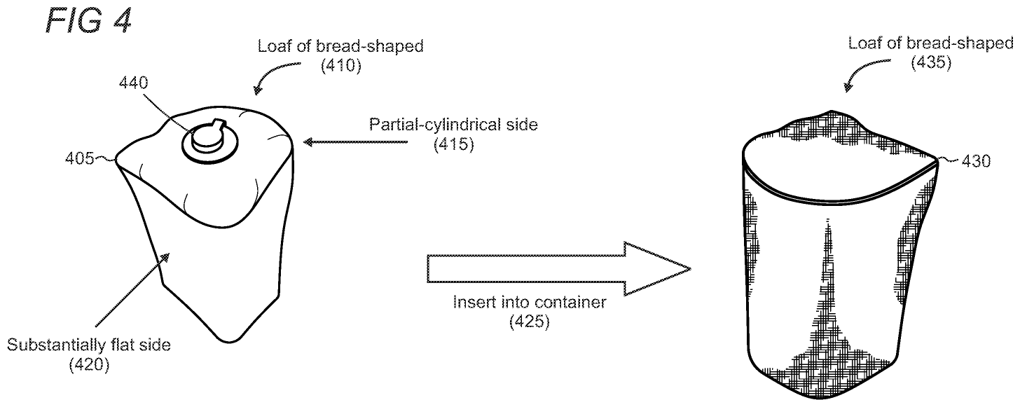

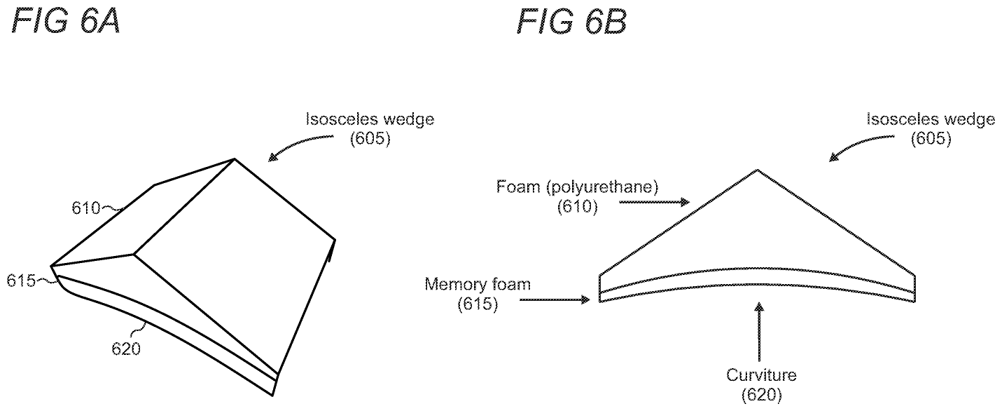

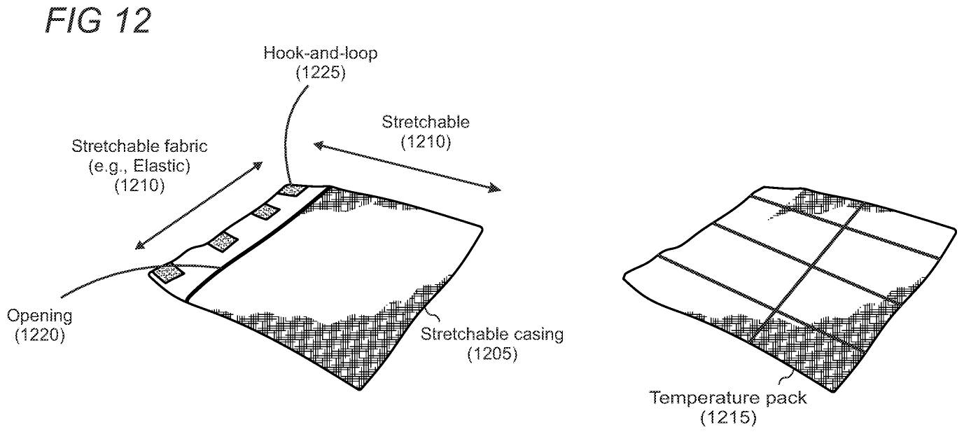

The maternity belt 105 can be used with a variety of cushions and components to provide specific forms and options of positional therapy for a user. The remainder of the drawings, FIGS. 4-17, illustrate various components that interoperate with the maternity belt, namely the central section loop fastener 150. In addition, the various cushions, pillows, and components shown in FIGS. 4-17 may be comprised of breathable, antimicrobial, and anti-odor materials. While a casing is shown with respect to the air chamber in FIG. 4 and the temperature pack in FIG. 12, each pillow and component may have a correspondingly sized and shaped casing inside which the component inserts. Each casing is used, in particular, for it's stretchable fabric and material and for having a corresponding hook-and-loop fastener to attach to the central section's hook-and-loop fastener 150. Thus, while FIGS. 6A-B, 10A-B, and 14 do not show a casing, each component may nonetheless have a casing that is sized and shaped to snug fit with the respective inserted component.

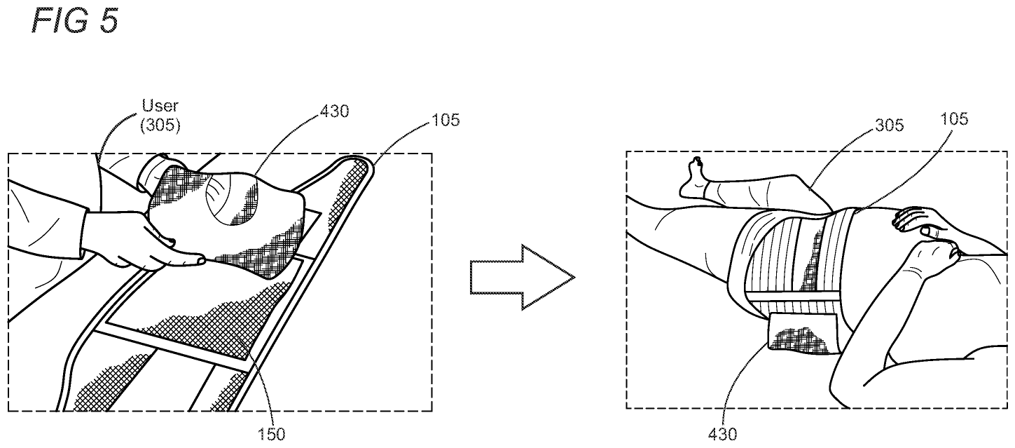

FIGS. 4 and 5 show illustrative representations in which an inflatable air chamber 405 can be utilized with the maternity belt 105. The air chamber is shaped like a loaf of bread 410, such that it has a partially cylindrical side 415 and a substantially flat side 420. The flat surface helps secure and maintain the air chamber's position when attached to the maternity belt's fastener 150. The air chamber may be inflated and deflated using the top 440, which opens to an inlet/outlet for the air chamber.

The air chamber 405 comes with a casing 430 that substantially corresponds in shape and size to the inflatable air chamber 405. The casing is likewise a loaf of bread-shaped 435 and can receive the air chamber when inflated. In addition, the casing, namely the substantially flat side of the casing, may be configured with the corresponding fastener to enable the inflatable air chamber to attach to the maternity belt's fastener.

FIG. 5 shows an illustrative representation in which user 305 attaches the casing 430, which houses the inflatable air chamber 405, against the maternity belt's fastener 150. Once the component is attached, the user can put on the maternity belt (FIG. 3) and then lay down. Thus, the attachment and maternity belt together help the user achieve a desirable and safe position during rest or sleep.

FIGS. 6A and 6B show illustrative representations of an isosceles triangle wedge-shaped pillow 605. The wedge includes an upper portion comprised of a polyurethane foam 610 and a lower portion comprised of a memory foam 615. The bottom portion of each layer includes a slight curvature 620 that extends inward to the wedge. The curvature is meant to accommodate a user's back shape when the wedge is attached to the maternity belt 105 and in use.

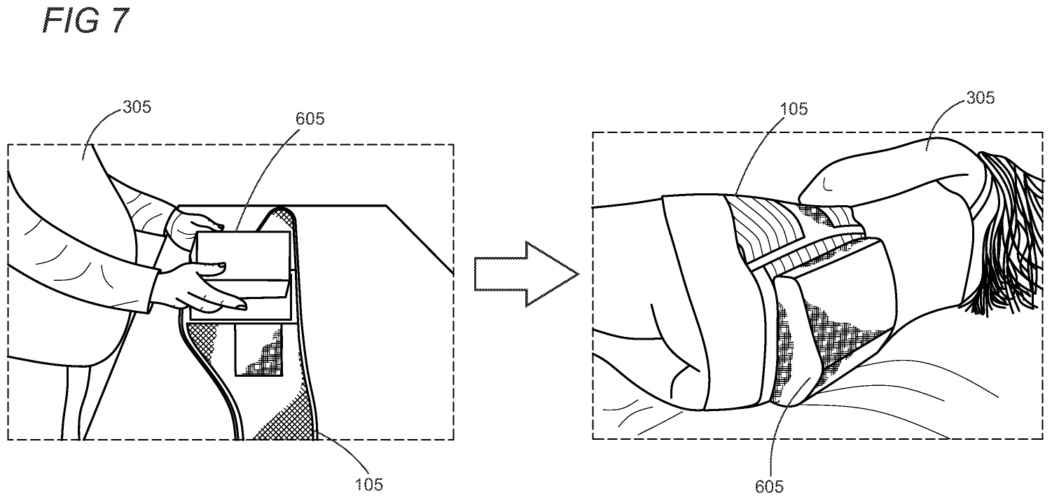

FIG. 7 shows an illustrative representation in which user 305 secures the isosceles triangle wedge-shaped pillow 605 to the maternity belt's fastener 150. Once attached and the user puts on the maternity belt (FIG. 3), the user lays on their side. The isosceles triangle wedge-shaped pillow provides additional positional therapy support for the side position and prevents the user from lying flat on her back.

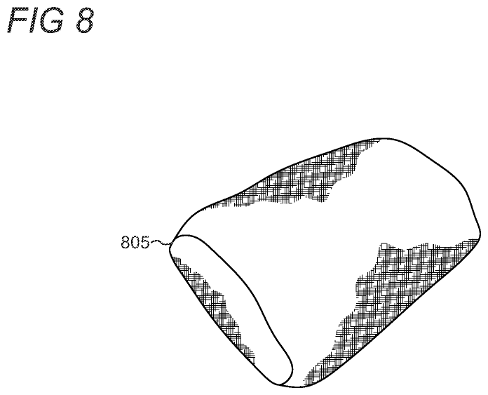

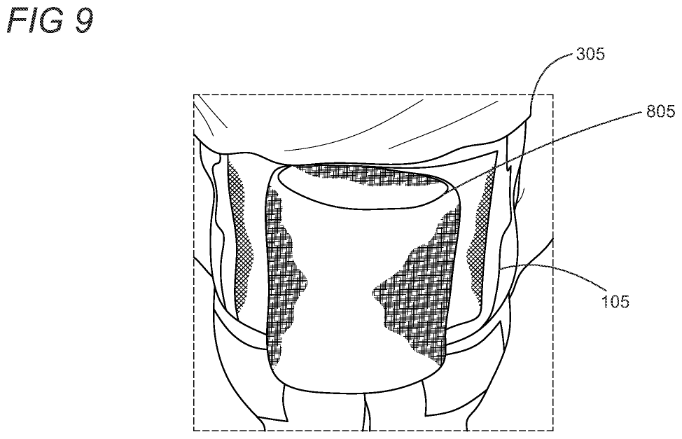

FIGS. 8 and 9 show illustrative representations of a bean bag pillow 805 that provides functional obstruction to the user's pelvis, which prevents the user from sleeping flat on her back. The beanbag pillow has one side that is substantially flat to provide a more secure and easier attachment to the maternity belt's fastener 150. Likewise, the bean bag pillow includes a corresponding fastener, such as a hook-and-loop fastener, to attach to the belt. As shown in FIG. 9, the bean bag pillow may be oriented vertically over the user's spine to provide the necessary positional therapy and avoid the user from lying flat on her back. FIGS. 8 and 9 show the bean bag pillow positioned inside a casing. Similar to the other components discussed herein, the bean bag pillow may be comprised of a foam material that is inserted into the correspondingly shaped and sized casing.

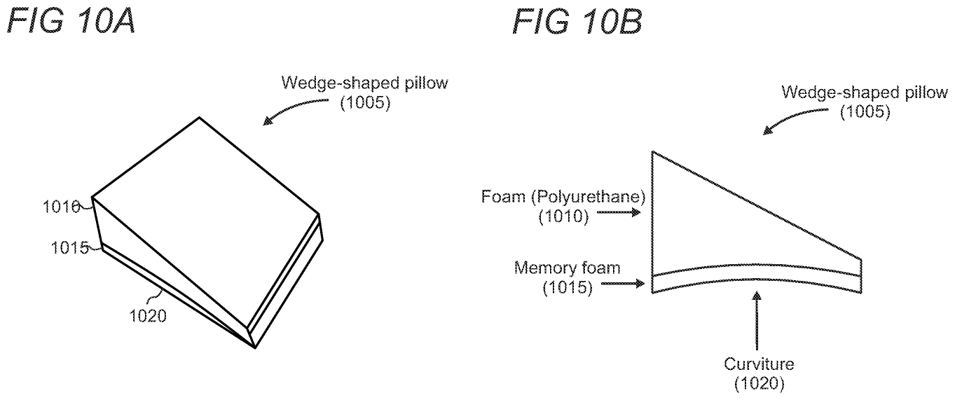

FIGS. 10A and 10B show illustrative representations of a right-angle triangle wedge-shaped pillow 1005. The right-angle triangle wedge-shaped pillow is restrictive such that it provides functional obstruction to the user's pelvis, which prevents the user from sleeping flat on their back and keeps the pelvis tilted towards the left or right side. In addition, the attachment enables the user to select whether they want left side and left-pelvic-tilt sleep or right side and right-pelvic-tilt sleep.

The right-angle triangle wedge-shaped pillow 1005 is comprised of a foam polyurethane 1010 layer and a memory foam 1015 layer. In addition, the bottom of the pillow includes a curvature 1020 to provide greater comfort against the user's back when the pillow is secured to the maternity belt 105 and in use.

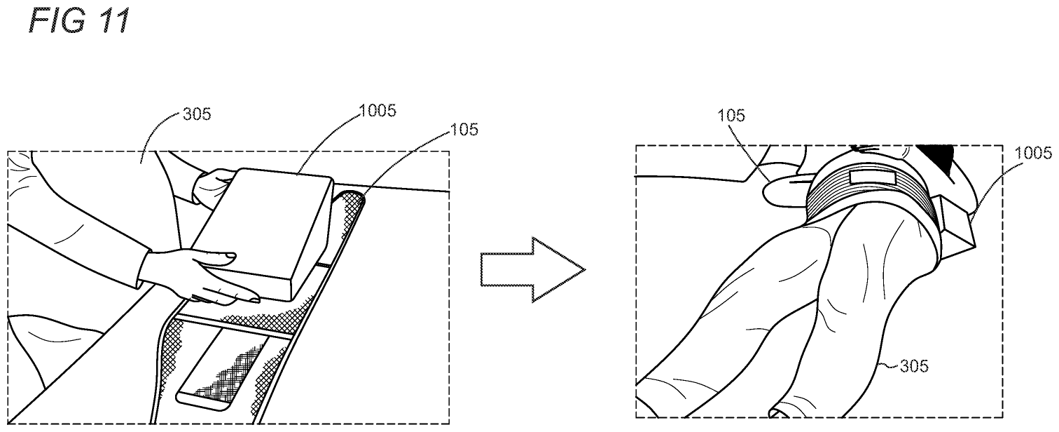

FIG. 11 shows an illustrative representation in which user 305 secures the right-angle triangle wedge-shaped pillow 1005 to the maternity belt's fastener 150. Once attached and the user puts on the maternity belt (FIG. 3), the user lays down on an angle. The right-angle triangle wedge-shaped pillow provides additional positional therapy support for the side position and prevents the user from lying flat on her back.

FIG. 12 shows an illustrative representation of a temperature pack 1215 and a stretchable casing 1205, which provide thermal therapy for musculoskeletal aches and pains related to pregnancy. The temperature pack can be a heated pack or an ice pack, depending on the scenario. The temperature pack can be inserted inside the casing's opening 1220. The casing 1205 is comprised, at least in part, of elastic and stretchable fabric, as representatively shown by numerals 1210. The casing is stretchable to stretch with the maternity belt as the bands or cinching straps are tightened, thereby preventing the casing and temperature pack from restricting the maternity belt's adjustability. In addition, the casing includes a hook-and-loop fastener 1225 to engage with the maternity belt's fastening mechanism 150.

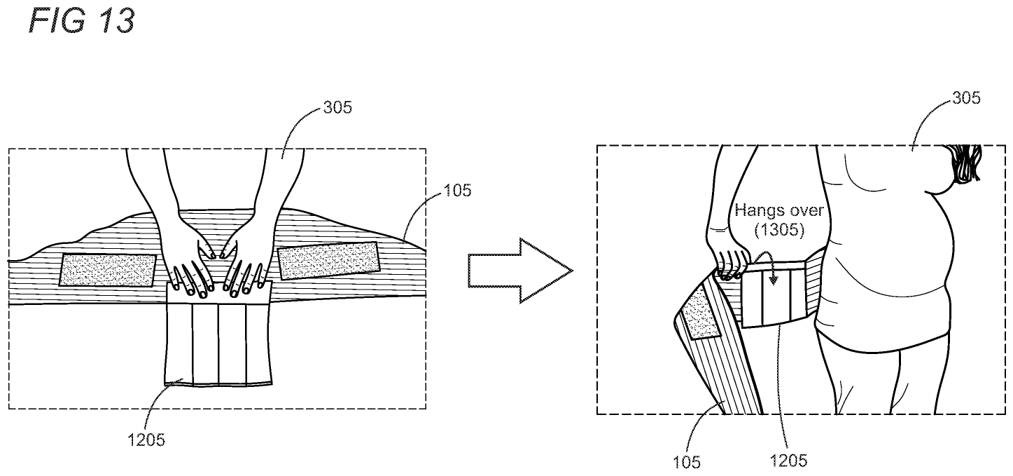

FIG. 13 shows an illustrative representation in which user 305 applies the hook-and-loop fastener 1225 of the stretchable casing (with the temperature pack 1215 inserted therein) to the maternity belt's corresponding fastener. The upper portion of the casing is attached to the maternity belt's exterior side fastener and then hangs over to the belt's interior so that the temperature pack and casing can directly engage with the user's back, as representatively shown by numeral 1305.

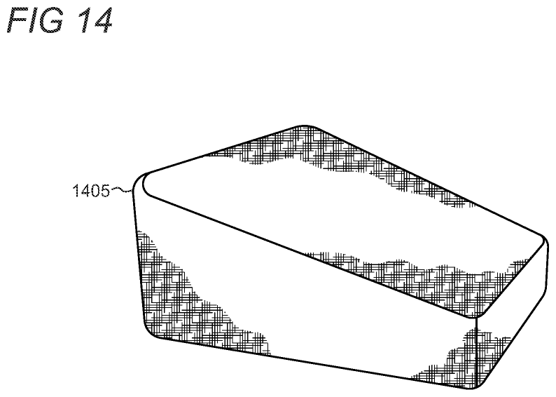

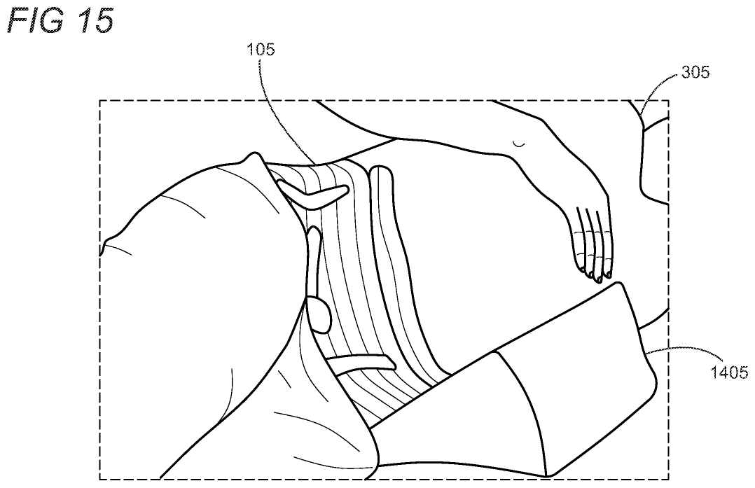

FIGS. 14 and 15 show illustrative representations of a trapezoidal side pillow 1405, which provides additional comfort to the user while sleeping. The trapezoidal side pillow can be placed under the user's abdomen when sleeping on the side to support the abdomen and relieve stress from the lower back. In typical implementations, the trapezoidal side pillow may not engage with the maternity belt's fastener 150 but is placed underneath the user's side, as shown in FIG. 15. Thus, the trapezoidal side pillow may or may not have a side with a hook-and-loop fastener as it likely would not come into contact with the maternity belt's fastener.

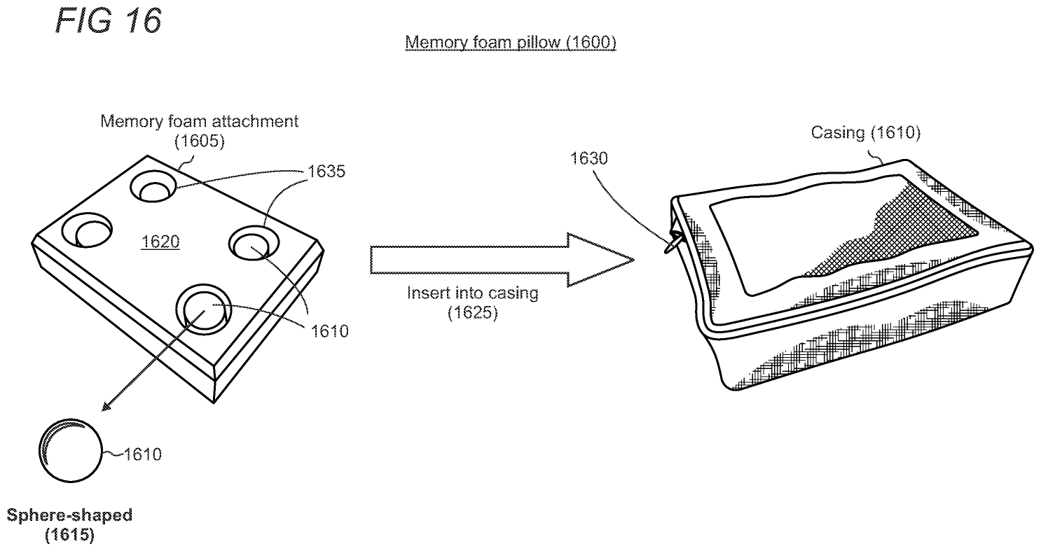

FIGS. 15 and 16 show illustrative representations in which a rectangular memory foam pillow 1600 with embedded firm shapes 1610 are utilized with the maternity belt 105. The pillow is composed of polyurethane memory foam 1620 and the balls are comprised of polyprophylene. The memory foam attachment 1605 component is inserted into the casing 1610 to form the memory foam pillow 1600. The casing is utilized for its corresponding hook-and-loop fastener that attaches to the fastener on the maternity belt's fastener (FIG. 1).

The memory foam pillow 1600 includes a sets of two holes 1635 at the right and left edge that extend entirely from the top to the bottom side. A firm spherical shape is placed in each hole as representatively shown by numeral 1615, but other shapes are also possible. The holes are present to help maintain the relative positions of the firm spherical shapes and prevent them from escaping or moving during use. A user may be at least partially lying flat on their back when using the maternity belt 105 with the rectangular memory foam pillow with embedded firm shapes attached. The firm spherical shapes are positioned along the central section to provide subtle pressure points at specific anatomically relevant locations when the user is lying flat on their back, which prompts the user to change positions to their side.

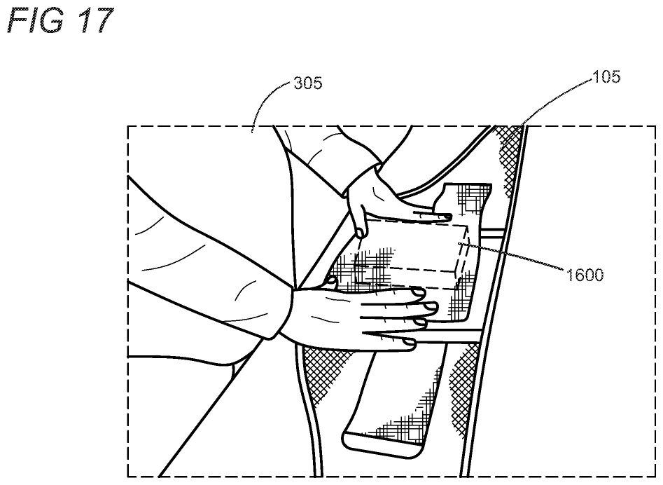

FIG. 17 shows an illustrative representation in which user 305 secures the rectangular memory foam pillow 1600 with embedded firm shapes 1610 to the maternity belt's fastener 150. Once attached, the user can put the maternity belt on (FIG. 3) and then lay down. The memory foam pillow with embedded firm shapes provides positional therapy by providing subtle pressure points at specific anatomically relevant locations on the user when she is flat on her back, which prompts her to change her position to her side.

Various embodiments are disclosed herein. In one exemplary embodiment, disclosed is a women's maternity belt having an exterior side and an interior side, the maternity belt being adapted to provide positional therapy during sleep, comprising: a right band having a distal end and a central section end; a left band having a distal end and a central section end; and a central section positioned in between the right and left bands, wherein the exterior side of the central section includes an attachment mechanism.

As another example, the attachment mechanism is a hook-and-loop fastener. As a further example, further comprising an inflatable air chamber positioned inside a correspondingly-shaped casing, wherein the inflatable air chamber and casing are substantially cylindrical with one substantially flat side, and wherein the substantially flat side of the casing includes a hook-and-loop fastener to attach to the maternity belt's hook-and-loop fastener. As another example, further comprising a bean bag pillow and correspondingly-shaped casing, an isosceles wedge-shaped pillow and correspondingly-shaped casing, a right-angle wedge-shaped pillow and correspondingly-shaped casing, a rectangular memory foam pillow with embedded firm shapes and correspondingly-shaped casing, a temperature pack and corresponding casing, and a trapezoidal side pillow and correspondingly-shaped casing, each one of which includes a hook-and-loop fastener on the casing to attach to the maternity belt's hook-and-loop fastener. In another example, further comprising a temperature pack and correspondingly-shaped casing inside which the temperature pack is inserted, wherein the casing is comprised of elastic material and stretchable to enable the casing to stretch with the maternity belt when the left band or right band are stretched during use. In another example, further comprising a bean bag pillow positioned inside a correspondingly-shaped casing, wherein the bean bag pillow and casing are substantially rectangular with one substantially flat side, and wherein the substantially flat side of the casing includes a hook-and-loop fastener to attach to the maternity belt's hook-and-loop fastener. In another example, further comprising an isosceles triangle wedge-shaped pillow positioned inside a correspondingly-shaped casing, wherein a bottom surface of the isosceles triangle wedge-shaped pillow and casing is inwardly curved to accommodate a back of a user when attached to the maternity belt's hook-and-loop fastener and worn by a user, and wherein the inwardly curved side of the casing includes a hook-and-loop fastener to attach to the maternity belt's hook-and-loop fastener. As a further example, further comprising a right-angle wedge-shaped pillow positioned inside a correspondingly-shaped casing, wherein a bottom surface of the right-angle wedge-shaped pillow and casing is inwardly curved to accommodate a back of a user when attached to the maternity belt's hook-and-loop fastener and worn by a user, and wherein the inwardly curved side of the casing includes a hook-and-loop fastener to attach to the maternity belt's hook-and-loop fastener. As a further example, further comprising a memory foam attachment with embedded firm shapes positioned, the memory foam attachment positioned inside a correspondingly-shaped casing to form a memory foam pillow, wherein the casing is substantially rectangular with one substantially flat side, and wherein the substantially flat side of the casing includes a hook-and-loop fastener to attach to the maternity belt's hook-and-loop fastener. In another example, wherein the memory foam attachment includes multiple holes inside which respective spheres are positioned to provide pressure points during use of the maternity belt. As another example, the holes extend fully from a top side to a bottom side of the memory foam attachment. In another example, further comprising left and right cinching straps positioned on an exterior side of the maternity belt and which respectively overlap with the left and right bands, wherein the cinching straps are elastic and provide tightening and loosening of the maternity belt when worn. In another example, a surface of the left band's exterior side is comprised of a fastening material. In another example, the distal end of the right band's interior side is comprised of a fastening material that engages with the fastening material on the left band's exterior side.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A women's maternity belt having an exterior side and an interior side, the maternity belt being adapted to provide positional therapy during sleep, comprising:
a right band having a distal end and a central section end;
a left band having a distal end and a central section end; and
a central section positioned in between the right and left bands, wherein the exterior side of the central section includes an attachment mechanism.

2. The women's maternity belt of claim 1, wherein the attachment mechanism is a hook-and-loop fastener.

3. The women's maternity belt of claim 2, further comprising an inflatable air chamber positioned inside a correspondingly-shaped casing, wherein the inflatable air chamber and casing are substantially cylindrical with one substantially flat side, and wherein the substantially flat side of the casing includes a hook-and-loop fastener to attach to the maternity belt's hook-and-loop fastener.

4. The women's maternity belt of claim 2, further comprising a bean bag pillow and correspondingly-shaped casing, an isosceles wedge-shaped pillow and correspondingly-shaped casing, a right-angle wedge-shaped pillow and correspondingly-shaped casing, a rectangular memory foam pillow with embedded firm shapes and correspondingly-shaped casing, a temperature pack and corresponding casing, and a trapezoidal side pillow and correspondingly-shaped casing, each one of which includes a hook-and-loop fastener on the casing to attach to the maternity belt's hook-and-loop fastener.

5. The women's maternity belt of claim 2, further comprising a temperature pack and correspondingly-shaped casing inside which the temperature pack is inserted, wherein the casing is comprised of elastic material and stretchable to enable the casing to stretch with the maternity belt when the left band or right band are stretched during use.

6. The women's maternity belt of claim 2, further comprising a bean bag pillow positioned inside a correspondingly-shaped casing, wherein the bean bag pillow and casing are substantially rectangular with one substantially flat side, and wherein the substantially flat side of the casing includes a hook-and-loop fastener to attach to the maternity belt's hook-and-loop fastener.

7. The women's maternity belt of claim 2, further comprising an isosceles triangle wedge-shaped pillow positioned inside a correspondingly-shaped casing, wherein a bottom surface of the isosceles triangle wedge-shaped pillow and casing is inwardly curved to accommodate a back of a user when attached to the maternity belt's hook-and-loop fastener and worn by a user, and wherein the inwardly curved side of the casing includes a hook-and-loop fastener to attach to the maternity belt's hook-and-loop fastener.

8. The women's maternity belt of claim 2, further comprising a right-angle wedge-shaped pillow positioned inside a correspondingly-shaped casing, wherein a bottom surface of the right-angle wedge-shaped pillow and casing is inwardly curved to accommodate a back of a user when attached to the maternity belt's hook-and-loop fastener and worn by a user, and wherein the inwardly curved side of the casing includes a hook-and-loop fastener to attach to the maternity belt's hook-and-loop fastener.

9. The women's maternity belt of claim 2, further comprising a memory foam attachment with embedded firm shapes positioned, the memory foam attachment positioned inside a correspondingly-shaped casing to form a memory foam pillow, wherein the casing is substantially rectangular with one substantially flat side, and wherein the substantially flat side of the casing includes a hook-and-loop fastener to attach to the maternity belt's hook-and-loop fastener.

10. The women's maternity belt of claim 9, wherein the memory foam attachment includes multiple holes inside which respective spheres are positioned to provide pressure points during use of the maternity belt.

11. The women's maternity belt of claim 10, wherein the holes extend fully from a top side to a bottom side of the memory foam attachment.

12. The women's maternity belt of claim 1, further comprising left and right cinching straps positioned on an exterior side of the maternity belt and which respectively overlap with the left and right bands, wherein the cinching straps are elastic and provide tightening and loosening of the maternity belt when worn.

13. The women's maternity belt of claim 1, wherein a surface of the left band's exterior side is comprised of a fastening material.

14. The women's maternity belt of claim 13, wherein the distal end of the right band's interior side is comprised of a fastening material that engages with the fastening material on the left band's exterior side.
